(19) United States
(12) Patent Application Publication
(10) Pub. No.: US 2005/0197699 A1
(43) Pub. Date: Sep. 8, 2005

(54) Tissue repair apparatus and method

(76) Inventors: Daniel Irwin Jacobs, Palo Alto, CA (US); Robert Elson, Los Altos Hills, CA (US); Kyle Naydo, Mountain View, CA (US); Russell Layton, Sunnyvale, CA (US)

Correspondence Address: DLA PIPER RUDNICK GRAY CARY US, LLP, 2000 UNIVERSITY AVENUE, E. PALO ALTO, CA, 94303-2248, US

(21) Appl. No.: 11/056,735

(22) Filed: Feb. 10, 2005

Related U.S. Application Data
(60) Provisional application No. 60/543,533, filed on Feb. 10, 2004.

(51) Int. Cl. A61F 2/08

(52) U.S. Cl. 623/13.14; 623/13.18

(57) ABSTRACT

One or more tine plates for repairing tissue (e.g. hand/wrist tendons). Each plate has a center portion includes fenestrations, and/or a width or thickness that is less than that of first and second end portions of the plate, such that the center portion is more pliable than the first and second end portions. A first plurality of tines extends from the first end portion non-orthogonally and angled toward the center portion, and a second plurality of tines extends from the second end portion non-orthogonally and angled toward the center portion. A suture, button or ring are used to affix the plate(s) to the tendon, which the plate(s) extending across a sever point to serve as the load-bearing member, wherein the tines are positioned on both sides of the tendon sever point for fixating the tissue.

This application claims the benefit of U.S. Provisional Application No. 60/543,533, filed Feb. 10, 2004.

### FIELD OF THE INVENTION

The present invention relates to the repair of severed tendons, and more particularly to a method and apparatus for the repair of tendons of the hand and wrist.

### BACKGROUND OF THE INVENTION

Presently it can be difficult to repair tissue such as tendons in the hand and wrist that become severed or deeply lacerated (e.g. a cut 50% or more through the tissue) through accident or injury. Especially difficult to repair are the flexor tendons, which are the tendons that enable one to close their hand, when severed at the fingers. The flexor tendons in this area have a generally elliptical cross section, and travel through membrane-like sheaths and sinewy-like tunnels that are the equivalent of a human pulley system that hold the tendon close to the bone and make it possible to bend the fingers at the joints through muscle constrictions in the arm. These tendons have a pliable composition that does not uniformly hold that cross-section shape, especially as the tendon goes through the tendon pulleys.

When such tendons become severed, any medical solution or device that is used to repair the wound must securely hold severed section of tendon together so the tendon can heal together and reform a biological bond. The medical solution also needs to be contained within the tendon sheath and be able to travel through the tendon pulleys in the hand (i.e. must be sufficiently compliant and flexible). Ideally, there should be sufficient strength in the repair to enable early mobility of the hand and wrist for rehabilitation while providing an environment for the wound to heal. The medical solution also ideally needs to work with short pieces of exposed tendon tissue to minimize additional trauma to wound area to implement the repair, and needs to minimize necrosis of the tissue that would impair the healing process.

The use of sutures is the current prevalent solution for tendon repair and several suturing techniques have been developed by many individuals, which are highly skilled operations, in attempt to provide the repair strength required without causing excessive bulk that would impede travel through the tendon pulleys. The success of the repair is measured not only on the ability to bring the severed ends together but also the ability to go through a post operative rehabilitation program without rupturing while regaining as much mobility of the hand and wrist as there was before the tendon was severed. Failure of the repair is the result of insufficient number of sutures traversing the repair site and pulling through the tendon tissue. The greater mode of failure seen is the suture knots slipping and becoming untied when under load. Limited surgical working environment can make the procedure difficult to implement the loops of suture (core strands) through the tendon ends to provide an adequate tendon repair.

Another proposed solution is disclosed in U.S. Pat. No. 6,712,830 issued to Esplin, where a pair of anchors are engaged with the tissue on each side of the repair site. Each anchor includes teeth for engaging the tissue. The anchors are held to the tissue by sutures, and sutures are also used to longitudinally pull the anchors toward each other to draw the repair surfaces together. However, since the load-bearing member of the repair is still core strands of suture across the severed ends, it still possesses current modes of failure as with the before mentioned suture repair solutions.

U.S. Pat. No. 6,645,226 issued to Jacobs discloses a multi-point distribution system for tissue approximation. However, this patent does not contemplate optimizing the flexibility of one portion of the system relative to other portions thereof.

There is a need for a tendon repair apparatus and method that reliably secures severed ends of tendon together, allows early mobility of the hand and wrist to promote proper healing of these tendon ends together, and simplifies the repair procedure.

### SUMMARY OF THE INVENTION

The present invention solves the aforementioned problems by providing a tissue approximation assembly that securely and reliably fixates the ends of severed tendons together. The tissue approximation assembly is relatively easy to implement, and works well with relatively short sections of tendon tissue.

The tissue repair device of the present invention includes a first plate having a center portion disposed between first and second end portions, wherein the center portion includes at least one of fenestrations, a width that is less than that of the first and second end portions, and a thickness that is less than that of the first and second end portions, such that the center portion is more pliable than the first and second end portions, a first plurality of tines extending from the first end portion in a non-orthogonal manner, wherein the first plurality of tines are angled toward the center portion, and a second plurality of tines extending from the second end portion in a non-orthogonal manner, wherein the second plurality of tines are angled toward the center portion.

The method of repairing tissue having a sever point defining repair surfaces to be held together for healing of the present invention includes placing a first plate across the sever point, pressing the plate against the tissue, and securing the plate to the tissue. The first plate includes a center portion disposed between first and second end portions, wherein the center portion includes at least one of fenestrations, a width that is less than that of the first and second end portions, and a thickness that is less than that of the first and second end portions, such that the center portion is more pliable than the first and second end portions, a first plurality of tines extending from the first end portion in a non-orthogonal manner, wherein the first plurality of tines are angled toward the center portion, and a second plurality of tines extending from the second end portion in a non-orthogonal manner, wherein the second plurality of tines are angled toward the center portion. The pressing of the plate against the tissue is performed such that the first plurality of tines penetrates into and fixates the tissue on one side of the sever point and the second plurality of tines penetrates into and fixates the tissue on another side of the sever point. The securing of the plate to the tissue is performed such that the repair surfaces are held together by the first and second pluralities of tines.

Other objects and features of the present invention will become apparent by a review of the specification, claims and appended figures.

### BRIEF DESCRIPTION OF THE DRAWINGS

FIG. 1 is a perspective view of the tissue approximation assembly of the present invention, illustrating the top and bottom tine plates thereof.

FIG. 2 is a perspective view of the tissue approximation assembly of the present invention, illustrating how the top and bottom tine plates can be positioned to engage a tendon therebetween.

FIG. 3 is a side view illustrating the angle of the tines.

FIG. 4 is a side cross-sectional view illustrating the tissue approximation assembly of the present invention engaged with tendon tissue.

FIG. 5 is a side view illustrating a single plate embodiment of the present invention engaged with tendon tissue.

FIG. 6A illustrates buttons used to hold the tissue approximation plates together.

FIG. 6B illustrates rings used to hold the tissue approximation plates together.

FIG. 7A is a perspective view of the tissue approximation assembly of the present invention, illustrating the center portion of reduced width.

FIG. 7B is a perspective view of the tissue approximation assembly of the present invention, illustrating fenestrations in center portion.

### DETAILED DESCRIPTION OF THE PREFERRED EMBODIMENTS

The present invention is a tissue approximation assembly, and a method of implementing the same, that securely and reliably fixates two sections of tissue together while preserving mobility and promoting healing. The present invention is described in the context of fixating sections of flexor tendons of the hand together, but the disclosed apparatus is neither limited to the tendons of the hand nor tendon tissue in general and any appropriate tissue can be fixated together using the approximation assembly of the present invention.

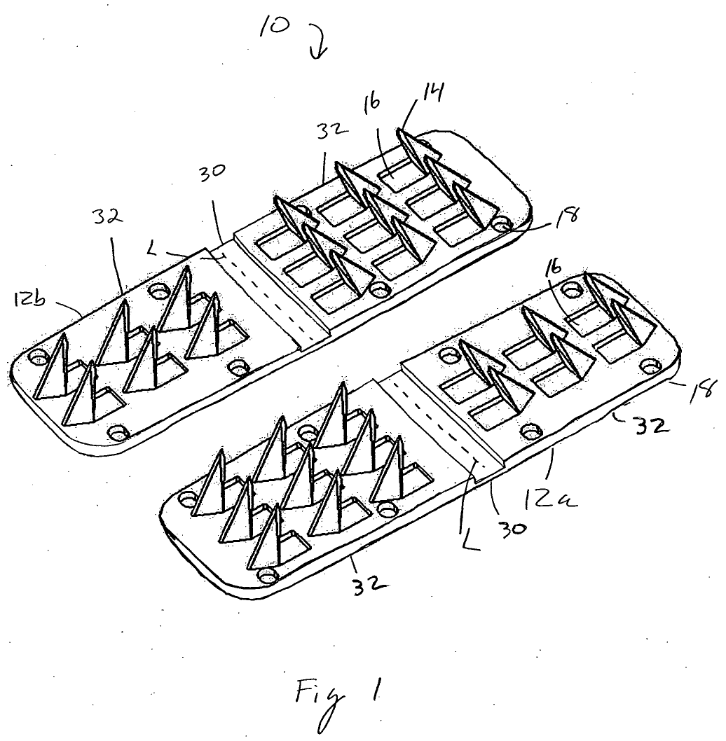

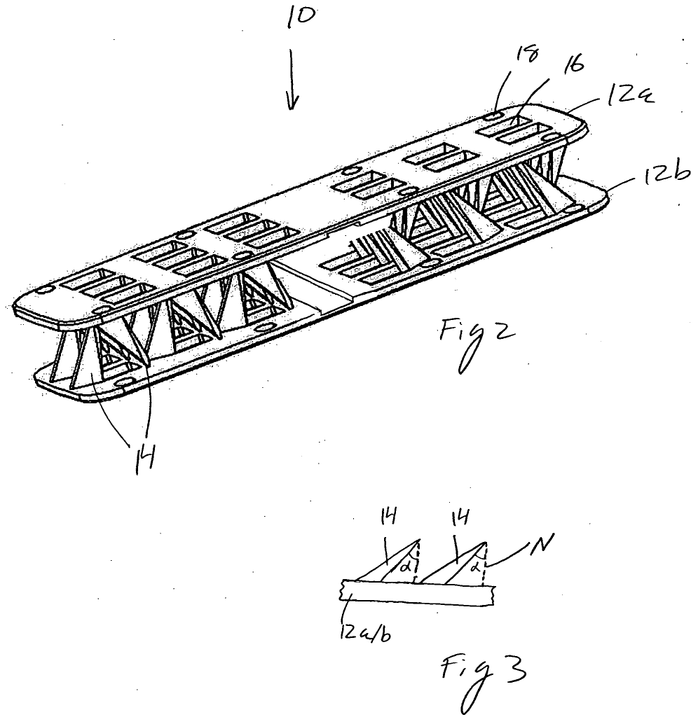

FIGS. 1 and 2 illustrate the approximation assembly 10 of the present invention, which includes top and bottom tine plates 12a/12b configured to engage with opposing sides of a severed tendon. Tine plates 12a/12b may be made of any appropriate biocompatible material, and preferably a biocompatible material which also may be biodegradable. Each of the tine plates 12a/12b is generally flat, and includes a plurality of tines 14 extending therefrom. Fenestrations (apertures) 16 are formed through plates 12a/12b (adjacent to each tine 14), to provide access for nutrients from synovial fluid in the tendon sheath for the tissue disposed between the tine plates 12a/12b. In addition, suture holes 18 are formed through plates 12a/12b adjacent the peripheries thereof for securing the plates to the tendon.

Tines 14 extend from each of the plates 12a/12b at a non-orthogonal (i.e. non-normal) manner. Each plate 12a/12b includes two sets (pluralities) of tines, each on opposite sides of the centerline L of the plate. For each tine set, the tines 14 are angled toward the centerline L, by and angle α preferable between about 10 to 45 degrees relative to the normal N of the plate, as illustrated in FIG. 3. If the tine angle α is less than around 10 degrees from the plate normal N, the tissue may become disengaged from the tines 14 during use. If the tine angle α is greater than around 45 degrees relative to the plate normal N, it can become difficult to properly engage the tines with the tissue when attempting to press into it. Tine angle α as used herein refers to the angle of tine surface facing toward the centerline L, relative to the 90 degree normal N of the plate surface. The angle of the tine back surface is not as critical, and can be more or less than the tine angle α depending upon the shape of the tines. A tine angle α of around 18 degrees from the plate normal N has been found to work well for engaging with tendon tissue.

Each tine set is configured to engage with tissue, and to not interfere with the tine set from the other tine plate positioned on the opposite side of the tissue. Thus, as best shown the FIG. 1, an exemplary tine set configuration can be a 3×3 configuration of 9 tines on one side of the centerline L, and a 2×3 configuration of 6 tines on the other side of the centerline L, where the tine set of 6 tines of one plate can be oriented opposite to, and in between, the 9 tines of the tine set from the other plate (i.e. in an interlaced fashion, with opposing tines offset from each other), and vice versa, as best shown in FIG. 2. In this manner, each tine can fully engage with the tissue without interfering with any tines from the other plate. In the preferred embodiment shown in FIG. 1, tine plates 12a/12b are identical, but oriented so that the tine set of 6 tines from one plate is positioned opposite to the tine set of 9 tines from the other plate, and vice versa.

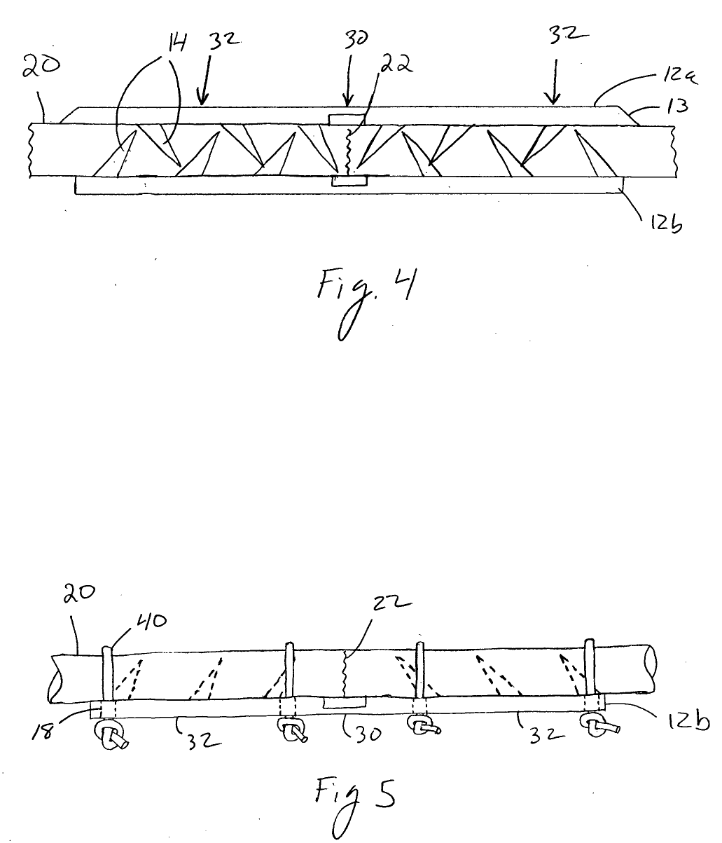

At or near the centerline L, each plate 12a/12b includes a center portion 30 having a thickness that is less than that of the end portions 32 of the plates 12a/12b to better flex (i.e. greater pliability) during use, which is important for allowing the plates 12a/12b to better travel through the bend radius of a closed finger and through the tendon pulleys in the human hand. Having a greater plate thickness in the plate end portions 32 ensures adequate structural support and rigidity for the tines 14. Thus, the combination of thickness difference (thinner plate at the center portion 30 relative to thicker plate at end portions 32) provides both flexibility of the plates with rigid support for the tines for superior performance. The edges and corners of the plates 12a/12b are preferably rounded and smooth, and plates 12a/12b are equal or narrower in width than the width of the tendon, to ensure better travel through the tendon sheath and pulleys. For the plate disposed on the volar side of the tendon (i.e. the side of the tendon that is facing the palm of the hand and runs against the pulley during finger closure), those ends of the plate can include a ramped side surface (i.e. angled side surface) so the plate enters the tendon pulleys with less interference, as illustrated on plate 12a in FIG. 4.

FIG. 4 illustrates how tine plates 12a/12b fixate tendon tissue 20 together. Tendon tissue 20 has a partial or complete sever point 22 (which defines repair surfaces that need to be held together for proper healing). Therefore, tine plates 12a/12b are placed on the opposing, flatter side surfaces of the tendon, extending across the repair surfaces. With the repair surfaces held against each other (at the sever point), the tine plates 12a/12b are pressed together from opposite sides of the tendon 20, where tines 14 penetrate into the tendon and secure the sever point together. The plates are then held in place by rings, buttons/snaps, sutures, etc. that exert forces orthogonal to the load on the tendon tissue. For example, suture(s) can be threaded through the suture holes 18 and pulled to maintain a fixed distance between the two plates 12a/12b. The suture holes 18 are preferably evenly spaced around the tine sets of the tine plates 12a/12b so that the tines stay securely penetrated in the tissue after implementation.

The present invention has many advantages: the load-bearing plates 12a/12b extend across the sever point 22 to better hold the repair surfaces together; the geometry of plates 12a/12b generally match the surface shape of the tendon for ideal fixation; the tines 14 engage the cross-sectional shape from opposite sides so that the tendon ends (repair surfaces) at the sever point 22 are held together for proper healing; fixation is achieved by pressing the tines into the tendons and maintaining the engagement, without requiring additional surgeon skill to manually suture the tendon ends together; fixation forces are spread out across the tine plates 12a/12b via the tines, and along the length of tissue fixated therebetween; the tine plates 12a/12b are flexible (given their planar and/or thin cross-sectional shape at the center portions 30), and thus will not adversely affect the movement of the tendon within its sheath and/or around the human pulleys within the hand; the apertures 16 allow nutrients to flow through the tine plates 12a/12b and to the tendon fixated therebetween; tine plates 12a/12b can be made of biodegradable material so that they naturally dissolve away after the tendon ends have formed a sufficiently strong biological bond across the sever point, and the compression force between the plates (and onto the tissue) necessary for tissue fixation by tines is not enough to cause necrosis in the tissue or prohibit tissue healing.

It should be noted that the size of plates 12a and 12b, and/or the size of the tines thereon, need not be the same. In fact, one plate can be omitted, so long as the remaining plate traverses the repair surfaces 22 being held together, includes a set of tines on each side of the repair surfaces angled toward the center portion 30 of the plate having a reduced thickness compared to end portions 32 for improved flexibility. FIG. 5 illustrates the use of a single tine plate 12a where sutures 40 extend around the tendon 20 and are secured to the suture holes 18. With a single plate embodiment, the tine plate 12a is ideally affixed to the dorsal side of the tendon, for better and smoother movement through the sheath. The sutures going around the tendon could actually pierce through a portion of the tendon to keep them fiom moving or sliding along the tendon.

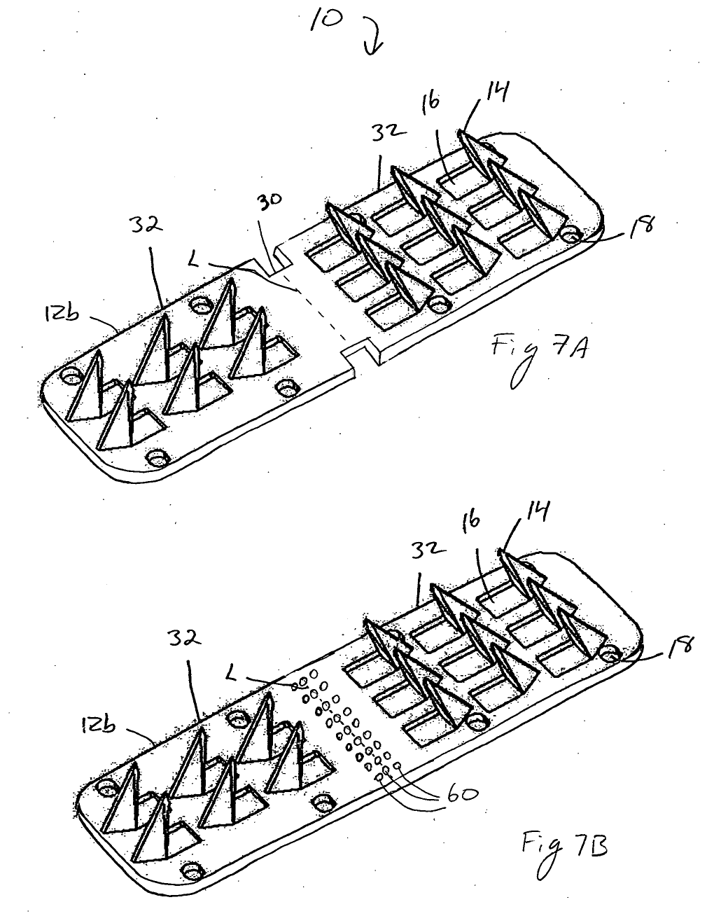

Ideally, the center portion 30 having reduced thickness as shown in the figures should not be too narrow, as it is preferable to have the tine plate flex over an area instead of a narrow line (to prevent material fatigue). In addition, reducing the thickness is not the only way to increase the pliability of center portion 30. Increased pliability of the center portion 30 can also be accomplished by reducing its width (as shown in FIG. 7A), forming fenestrations (holes) 60 that extend partially or fully through center portion 30 (as shown in FIG. 7B), and/or any combination of the above. The reduction in width/thickness, or the inclusion of the fenestrations, can be made gradually (i.e. a gradual reduction in the width and/or thickness dimensions or a gradual increase in fenestration frequency/density) to create a portion of plate 12 of greater pliability relative to others without creating a sudden change in pliability that could result in material fatigue.

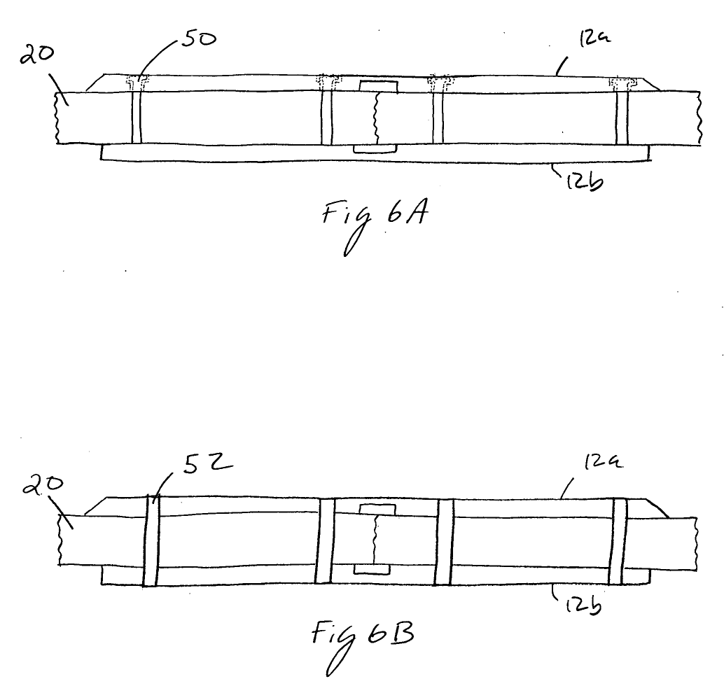

It is to be understood that the present invention is not limited to the embodiment(s) described above and illustrated herein, but encompasses any and all variations falling within the scope of the appended claims. For example, the shape, number and position of the tines 14, apertures 16 and suture holes 18 can vary. Suture holes 18 can be omitted in favor of other features such as v-slots or other irregularities to establish a better mechanical lock for the suture. Rings/wires, buttons/snaps or other clamping mechanisms can be used to hold the tine plates 12a/12b together and/or to the tissue instead of sutures through the suture holes, such as that shown in FIGS. 6A (buttons 50 snapping through apertures 18) and 6B (rings 52 slid over or otherwise clasped around plates 12a/12b), preferably in a flush manner to minimize any protrusions on the outside surfaces of the plates. The plates 12a/12b can be the same plate used twice or a two (or more) entirely different plates. The tines could be omitted from one of the plates, where the second plate would simply provide a platform for affixing the first plate to the tendon. Lastly, the tine plate can include one or more areas of reduced width/thickness and/or fenestrations (and thus greater pliability) between the rows of tines for better flexibility throughout the length of the tine plate (i.e. the plate could include a plurality of center portions of increased pliability, where the center portions are not at the exact center of the plate).

1. A tissue repair device comprising:
a first plate having a center portion disposed between first and second end portions, wherein the center portion includes at least one of fenestrations, a width that is less than that of the first and second end portions, and a thickness that is less than that of the first and second end portions, such that the center portion is more pliable than the first and second end portions;
a first plurality of tines extending from the first end portion in a non-orthogonal manner, wherein the first plurality of tines are angled toward the center portion; and
a second plurality of tines extending from the second end portion in a non-orthogonal manner, wherein the second plurality of tines are angled toward the center portion.

2. The tissue repair device of claim 1, wherein the first plate includes a plurality of apertures each extending through the first plate adjacent one of the tines.

3. The tissue repair device of claim 1, wherein the first plate is formed of a biodegradable material.

4. The tissue repair device of claim 1, wherein the first plate includes a plurality of apertures formed therethrough and located adjacent a periphery of the first plate.

5. The tissue repair device of claim 1, wherein each of the first and second plurality of tines has a surface facing toward the first plate center portion that extends from the first plate at an angle between about 10 and 45 degrees relative to a 90 degree normal of the first plate.

6. The tissue repair device of claim 1, wherein the first and second end portions each include a ramped side surface.

7. The tissue repair device of claim 1, further comprising:
a second plate having a second center portion disposed between third and fourth end portions thereof, wherein the second center portion includes at least one of fenestrations, a width that is less than that of the third and fourth end portions, and a thickness that is less than that of the third and fourth end portions, such that the second center portion is more pliable than the third and fourth end portions; and
means for securing the first plate to the second plate with tissue therebetween.

8. The tissue repair device of claim 7, wherein the securing means includes at least one of a suture, a button and a ring.

9. The tissue repair device of claim 7, wherein the second plate further comprises:
a third plurality of tines extending from the third end portion in a non-orthogonal manner, wherein the third plurality of tines are angled toward the second center portion; and
a fourth plurality of tines extending from the fourth end portion in a non-orthogonal manner, wherein the fourth plurality of tines are angled toward the second center portion.

10. The tissue repair device of claim 9, wherein the first and second plurality of tines are offset from the third and fourth plurality of tines in an interlaced manner as the first and second plates are secured together by the securing means.

11. A method of repairing a tissue having a sever point defining repair surfaces to be held together for healing, the method comprising:
placing a first plate across the sever point, wherein the first plate includes:
a center portion disposed between first and second end portions, wherein the center portion includes at least one of fenestrations, a width that is less than that of the first and second end portions, and a thickness that is less than that of the first and second end portions, such that the center portion is more pliable than the first and second end portions,
a first plurality of tines extending from the first end portion in a non-orthogonal manner, wherein the first plurality of tines are angled toward the center portion, and
a second plurality of tines extending from the second end portion in a non- orthogonal manner, wherein the second plurality of tines are angled toward the center portion;
pressing the plate against the tissue such that the first plurality of tines penetrates into and fixates the tissue on one side of the sever point and the second plurality of tines penetrates into and fixates the tissue on another side of the sever point; and
securing the first plate to the tissue such that the repair surfaces are held together by the first and second pluralities of tines.

12. The method of claim 11, wherein the first plate includes a plurality of apertures each extending through the first plate adjacent one of the tines to provide access for nutrients for the tissue portions disposed adjacent the tines.

13. The method of claim 11, wherein the first plate includes a plurality of apertures formed therethrough and located adjacent a periphery of the first plate, and wherein the securing of the first plate includes securing a suture to the apertures that wraps around or through the tissue.

14. The method of claim I 1, wherein each of the first and second plurality of tines has a surface facing toward the first plate center portion that extends from the first plate at an angle between about 10 and 45 degrees relative to a 90 degree normal of the first plate.

15. The method of claim 11, further comprising:
placing a second plate across the sever point, wherein the securing of the first plate includes securing the first plate to the second plate with the tissue therebetween.

16. The method of claim 15, wherein the second plate includes a second center portion disposed between third and fourth end portions thereof, and wherein the second center portion includes at least one of fenestrations, a width that is less than that of the third and fourth end portions, and a thickness that is less than that of the third and fourth end portions, such that the second center portion is more pliable than the third and fourth end portions.

17. The method of claim 16, wherein the securing of the first and second plates to each other includes at least one of a suture, a button and a ring.

18. The method of claim 16, wherein:
the second plate further comprises a third plurality of tines extending from the third end portion in a non-orthogonal manner such that the third plurality of tines are angled toward the second center portion, and a fourth plurality of tines extending from the fourth end portion in a non-orthogonal manner such that the fourth plurality of tines are angled toward the second center portion; and
the method further comprises:
placing the second plate across the sever point, pressing the second plate against the tissue such that the third plurality of tines penetrates into and fixates the tissue on one side of the sever point and the fourth plurality of tines penetrates into and fixates the tissue on another side of the sever point; and
wherein after the securing of the first and second plates, the first and second plurality of tines are offset from the third and fourth plurality of tines in an interlaced manner.

19. The method of claim 11, wherein at least a portion of the tissue is contained within a sheath.

20. The method of claim 11, wherein the tissue is flexor tendon.
